(19) United States
(12) Patent Application Publication
(10) Pub. No.: US 2004/0156894 A1
(43) Pub. Date: Aug. 12, 2004

(54) Use of edible acids in fast-dispersing pharmaceutical solid dosage forms

(76) Inventors: Leon Paul Grother, Swindon (GB); Lisa Garrett, Swindon (GB); Karolyn Tapper, Bishopstone (GB)

Correspondence Address: Donald O. Nickey, Cardinal Health, Inc., 7000 Cardinal Place, Dublin, OH, 43017, US

(21) Appl. No.: 10/360,905

(22) Filed: Feb. 7, 2003

(51) Int. Cl. A61K 31/415; A61K 9/64; A61K 9/20

(52) U.S. Cl. 424/465; 514/406

(57) ABSTRACT

In order to reduce the disintegration time of solid, oral, fast-dispersing, lyophilized, pharmaceutical dosage forms, an edible acid such as citric acid is included in a composition used to produce said dosage forms where there is a pharmaceutically active ingredient having a low water solubility.

### TECHNICAL FIELD

This invention relates to oral, fast-dispersing, pharmaceutical solid dosage forms and is more particularly concerned with such dosage forms where the pharmaceutically active ingredient has a low water solubility. The invention is especially, but not exclusively, concerned with such solid dosage forms which contain a high loading of such a pharmaceutically active ingredient.

### DESCRIPTION OF THE PRIOR ART

Solid, fast-dispersing pharmaceutical dosage forms are, per se, well known. For example, a fast-dispersing dosage form is disclosed in GB-A-1548022 which describes a dosage form comprising a network of the pharmaceutically active ingredient and a water-soluble or water-dispersible carrier which is inert towards the active ingredient, the network having been obtained by subliming solvent from a composition in the solid state (i.e. freeze-drying or lyophilizing), the composition comprising the active ingredient and a solution of the carrier in the solvent.

Fast-dispersing dosage forms typically disintegrate within one to ten seconds of being placed in the oral cavity. By the term “fast-dispersing” as used herein is meant that the solid dosage form will disintegrate in water at 37° C. in ten seconds or less when tested by a procedure which is analogous to the Disintegration Test for Tablets, B.P. 1973, and which is described in the above-mentioned GB-A-1548022.

W097/06786 (and equivalent EP-A-0850050) discloses a method of producing a fast-dispersing dosage form of apomorphine, in which gelatin and mannitol are dispersed in water, apomorphine hydrochloride is added and the mix is homogenized to ensure dissolution of the drug, following which citric acid is added gradually with stirring to adjust the solution pH to 3.0, and then further water is introduced and the bulk mix is homogenized to ensure that dissolution is complete. Predetermined quantities (sometimes referred to as “wet fill units”) of this dispersion are dosed into pre-formed blister pockets and the product is then freeze dried (lyophilized) to produce the solid, fast-dispersing dosage forms. In such a procedure, the citric acid is added to maximize the chemical stability of apomorphine which is a basic drug known to exhibit optimal chemical stability in an acidic environment. It is also water-soluble in such an environment. As an alternative to using citric acid, the use of tartaric acid, phosphoric acid, hydrochloric acid and maleic acid is disclosed.

WO98/31368 (and equivalent EP-A-0954314) also discloses the use of citric acid to adjust the pH in a dosage form containing water-soluble apomorphine hydrochloride.

Similarly, WO98/42344 (and equivalent EP-A-0969842) discloses a fast-dispersing solid dosage form in which citric acid is present to provide chemical stabilization of buspirone hydrochloride.

Furthermore, oral, fast-dispersing pharmaceutical dosage forms are disclosed in WO96/26714 (and equivalent EP-A-0814770) where the anti-Parkinson's disease drug, selegiline, is used. The free base of selegiline is a volatile oil which can evaporate during the manufacturing process or from the finished product. Accordingly, an acid such as citric acid, tartaric acid, phosphoric acid, hydrochloric acid or maleic acid is added to shift the equilibrium towards the salt form of selegiline by lowering the pH of the solution.

WO98/35656 (and equivalent EP-A-0973506) discloses dosage forms containing ketoprofen and stearic acid. The stearic acid is added as a lipid along with a sweetener to taste-mask an unpleasant or bitter tasting drug. The lipid and the drug become associated so that, when the dosage form disperses in the mouth, the drug is prevented from coming into contact with the mucosa and the taste is thereby masked.

### BRIEF DESCRIPTION OF THE INVENTION

In complete contrast to the above-mentioned uses of acids in fast-dispersing solid dosage forms, we have now discovered unexpectedly that acids can be used to decrease the disintegration time of dosage forms which contain a substantially water-insoluble pharmaceutically active ingredient and which have unacceptably long disintegration times (typically more than ten seconds). This is a completely unexpected discovery because the previous uses of acids for pH modification and/or taste-masking have been used in dosage forms containing water-soluble drugs and/or in dosage forms where long disintegration times have not been a problem.

By the term “substantially water-insoluble pharmaceutically active ingredient” as used herein is meant a pharmaceutically active ingredient whose water solubility is so low that, for a given fast-dispersing dosage form, a major portion (more than 50%) of the active ingredient is suspended (as opposed to being in solution) in a suspension (wet fill unit) which is lyophilized to produce that dosage form. Thus, this depends not only on the water-solubility per se but also upon the amount (or loading) of the active ingredient used in the dosage form.

Thus, according to one aspect of the present invention, there is provided the use of an edible acid in an oral, fast-dispersing, lyophilized (freeze-dried) pharmaceutical solid dosage form containing a substantially water-insoluble pharmaceutically active ingredient and a gelatin-based carrier, for reducing the disintegration time of the solid dosage form (as compared to the same dosage form without the edible acid).

According to another aspect of the present invention, there is provided a method for reducing the disintegration time of a solid, fast-dispersing, lyophilized, pharmaceutical dosage form containing a substantially water-insoluble pharmaceutically active ingredient and a gelatin-based, water-dispersible carrier, said method comprising the step of including at least one edible acid in a composition containing said substantially water-insoluble pharmaceutically active ingredient and said gelatin-based, water-dispersible carrier prior to formation of said solid dosage form from said composition.

The edible acid may be any of the pharmaceutical acceptable acids such as citric acid, maleic acid, tartaric acid or hydrochloric acid, with citric acid being preferred. The amount of acid used is such as to reduce the disintegration time of the solid dosage form to less than 10 seconds, and may be in the range of 0.01 to 10% by weight, more preferably from 0.1 to 5% by weight, but is typically not greater than 1%, by weight, based on the weight of the composition which is lyophilized to produce the solid dry dosage form.

According to a further aspect of the present invention, there is provided a method for reducing the disintegration time of solid, fast-dispersing, lyophilized, pharmaceutical dosage forms, said method comprising the steps of:

(i) forming a composition comprising water, a substantially water-insoluble pharmaceutically active ingredient, a gelatin-based, water-dispersible carrier and at least one edible acid selected from citric acid, maleic acid, tartaric acid and hydrochloric acid and mixtures of any one or more of such acids;

(ii) introducing portions of said composition into individual pockets; and

(iii) lyophilizing said portions in said pockets so as to dry and solidify said portions whereby to produce said solid dosage forms which contain said substantially water-insoluble pharmaceutically active ingredient, said gelatin-based, water-dispersible carrier and said at least one edible acid.

According to a still further aspect of the present invention, there is provided a method for the preparation of the disintegration time of solid, fast-dispersing, lyophilized, pharmaceutical dosage forms, said method comprising the steps of:

(i) forming a composition comprising water, a substantially water-insoluble pharmaceutically active ingredient, a gelatin-based, water-dispersible carrier and at least one edible acid selected from citric acid, maleic acid, tartaric acid and hydrochloric acid and mixtures of any one or more of such acids;

(ii) introducing portions of said composition into individual pockets; and

(iii) lyophilizing said portions in said pockets so as to dry and solidify said portions whereby to produce said solid dosage forms which contain said substantially water-insoluble pharmaceutically active ingredient, and wherein said dosage forms a disintegration times less than the same dosage forms without the edible acid.

All aspects of the invention are particularly applicable to solid dosage forms containing a pharmaceutically active ingredient in an amount which has an unacceptably deleterious effect upon the disintegration time. Such pharmaceutically active ingredients may be selected from insoluble or sparingly soluble analgesics, antihistamines, antitussives, antibiotics, bronchodilators, cardiovascular drugs, central nervous systems drugs, decongestants etc.

Specific examples of some of these include rofecoxib, paracetamol, and piroxicam.

The invention is considered to be especially suitable for solid dosage forms where the pharmaceutically active ingredient is present in a relatively high proportion. For drugs with a low water-solubility, long disintegration times are a major problem to overcome when formulating at high loadings of the active ingredient. Long disintegration times can arise as a result of the reduced porosity of the dosage form. Accordingly, the present invention is particularly suitable for overcoming or mitigating this problem. With the present invention, it is considered possible to produce fast-dispersing dosage forms from 1000 mg wet fill units containing as much as 300 to 400 mg of active ingredient. This results in a very high loading of the active ingredient in the dosage form after freeze-drying. Because of this, the present invention also allows a reduction in the size of the dosage form in cases where a larger dosage form may be feasible but not commercially viable. However, the present invention may also provide useful reductions in disintegration times for drug loadings as low as 10% by weight of the solid dry dosage form.

The gelatin-based carrier (or matrix-forming excipient) may preferably be derived from gelatin and mannitol. At higher concentrations of these, a stronger dosage form is produced that tends to have a longer disintegration time than a dosage formed with a lower concentration of these components. In order to be handlable and withstand the rigors of packing and transport procedures, a solid dosage form needs to have sufficient strength. Reducing the gelatin/mannitol concentrations results in a shorter disintegration time but, in some cases, the concentrations of gelatin and mannitol required to give rapid disintegration are so low that the tensile strength of the solid dosage form is so low that it is easily damaged.

Where the disintegration time of the dosage form varies depending upon the composition holding time prior to formation of the dosage form, there are practical limitations. The gelatin levels can be reduced to a point where the disintegration time is, within limits, acceptable irrespective of the composition holding time. However, this may result in a dosage form that is too weak. The other option is to limit the composition holding time to one in which the dosage forms produced at the end of the batch still have an acceptably short disintegration time. However, this may result in unacceptable costs due to low batch yields. When an acid is present, we have found that there is no significant difference between the disintegration times of dosage forms dosed after 1 hour holding time and 25 hours holding time. Without the acid, there can be a substantial increase in the disintegration time of dosage forms produced after a 24 hour composition holding time.

Instead of using mannitol as described above, there may be used other sugars, e.g. dextrose, lactose, galactose and trehalose; cyclic sugars such as cyclodextrin; inorganic salts such as sodium phosphate, sodium chloride and aluminium silicates; and amino acids having from 2 to 12 carbon atoms such as glycine, L-alanine, L-aspartic acid, L-glutamic acid, L-hydroxyproline, L-isoleucine, L-leucine and L-phenylalanine.

The gelatin and/or other carrier-forming component (when used) may be incorporated into the composition prior to solidification. The carrier-forming agent(s) may be present in addition to a surfactant or to the exclusion of a surfactant. In addition to forming the carrier, the carrier-forming agent(s) may aid in maintaining the dispersion of the relatively insoluble pharmaceutically active ingredient within the solution or suspension.

Secondary components such as preservatives, anti-oxidants, surfactants, viscosity enhancers, colouring agents, flavouring agents, sweeteners or taste-masking agents may also be incorporated into the mix.

### DETAILED DESCRIPTION OF THE INVENTION

### Examples 1 and 2 and Comparative Examples 1 and 2

The ingredients listed in Table 1 were formulated into solid dosage forms containing a 300 mg dose of paracetamol (water solubility 14 mg/ml) or piroxicam (water solubility <1 mg/ml. The solid dosage forms were produced as follows: The gelatin and the mannitol were added to the purified water and heated to 60° C. while stirring to allow the gelatin to dissolve. Where applicable, the citric acid was added at this point. The mix was then allowed to cool to 25° C., at which point the mix was added to the drug gradually with stirring to create a smooth, fluid suspension. 1 g aliquots of this suspension were dosed into pre-formed blister pockets and frozen rapidly under nitrogen. The frozen product was then freeze dried to produce the solid dosage forms.

TABLE 1
(all amounts in % by weight)

| | Comparative | | Comparative | |
|---|---|---|---|---|
| Material | Example 1 | Example 1 | Example 2 | Example 2 |
| Gelatin | 2.50 | 2.50 | 2.50 | 2.50 |
| Mannitol | 1.90 | 1.90 | 1.90 | 1.90 |
| Purified Water | 65.60 | 65.10 | 65.60 | 65.10 |
| Citric Acid | — | 0.50 | — | 0.50 |
| Piroxicam | — | — | 30.00 | 30.00 |
| Paracetamol | 30.00 | 30.00 | — | — |

The disintegration times and tensile strengths (3 point bend test) of the dosage forms produced in these Examples are shown in Table 2 below. The test to obtain the disintegration times is as set forth in GB-A-1548022 (U.S. Pat. No. 4371516)

TABLE 2

| | | Tensile Strength |
|---|---|---|
| Example | Disintegration Time | (Nmm⁻²) |
| Comp. Ex. 1 | 13.40 | 0.82 |
| Example 1 | 3.10 | 0.61 |
| Comp. Ex 2 | 6.94 | 0.45 |
| Example 2 | 3.40 | 0.38 |

It will be noted that the disintegration time for the dosage form containing paracetamol without citric acid was substantially in excess of the acceptable upper limit of 10 seconds, whereas the disintegration time of the dosage form containing paracetamol and citric acid was a very acceptable time of 3.10 seconds, without an unacceptable loss of tensile strength. While the disintegration time for the dosage form containing piroxicam was shortened when citric acid was present, it will be noted that the disintegration time in the absence of citric acid was still acceptable.

### COMPARATIVE EXAMPLE 3 and Examples 3 to 6

Following a similar procedure to that described in the previous Examples, solid dosage forms containing 400 mg of paracetamol were produced using the ingredients listed in Table 3 below.

TABLE 3
(all amounts in % by weight)

| | Comp. | | | | |
|---|---|---|---|---|---|
| Material | Example 3 | Example 3 | Example 4 | Example 5 | Example 6 |
| Gelatin | 2.50 | 1.50 | 1.75 | 1.00 | 0.75 |
| Mannitol | 1.90 | 1.13 | 1.30 | 0.75 | 0.55 |
| Purified | 55.60 | 56.87 | 56.45 | 57.85 | 58.30 |
| Water | | | | | |
| Citric Acid | — | 0.50 | 0.50 | 0.40 | 0.40 |
| Paracetamol | 40.00 | 40.00 | 40.00 | 40.00 | 40.00 |

The disintegration times and tensile strengths obtained are illustrated in Table 4 below.

TABLE 4

| | | Tensile Strength |
|---|---|---|
| Example | Disintegration Time(s) | (Nmm⁻²) |
| Comp. Example 3 | 25.57 | 1.41 |
| Example 3 | 7.25 | 0.74 |
| Example 4 | 9.69 | 0.84 |
| Example 5 | 7.31 | 0.41 |
| Example 6 | 7.28 | 0.32 |

The results shown in Table 4 indicate that solid dosage forms containing a 400 mg dose of paracetamol can be produced successfully and that reducing the gelatin level from 1.5 to 0.75% by weight and the mannitol level from 1.13 to 0.55 % w/w did not have a very marked effect on the disintegration time.

### Examples 7 to 10 and Comparative Examples 4 and 5

Following a procedure similar to that described in Examples 1 and 2, solid dosage forms containing 10 wt % of rofecoxib (substantially water-insoluble: 0.004 mg/ml) were produced using the ingredients listed in Table 5. The results of testing are listed in Table 5 below.

TABLE 5
(all amounts in % by weight unless otherwise indicated)

| | | | Comp. | Comp. | | Example |
|---|---|---|---|---|---|---|
| Material | Example 7 | Example 8 | Example 4 | Example 5 | Example 9 | 10 |
| rofecoxib | 10.00 | 10.00 | 10.00 | 10.00 | 10.00 | 10.00 |
| Gelatin | 3.25 | 4.25 | 3.25 | 4.25 | 4.25 | 4.25 |
| Mannitol | 2.44 | 3.19 | 2.44 | 3.19 | 3.19 | 3.19 |
| Citric Acid | 0.92 | 1.02 | — | — | — | — |
| 7.6% HCl | — | — | — | — | 1.48 | — |
| Maleic Acid | — | — | — | — | — | 0.41 |
| Purified | 83.39 | 81.54 | 84.31 | 82.56 | 81.08 | 82.15 |
| Water | | | | | | |
| ¹TS (25 h, | 0.194 ± 0.006 | 0.261 ± 0.012 | 0.393 ± 0.032 | 0.818 ± 0.199 | 0.497 ± 0.048 | 0.389 ± 0.053 |
| 10° C.) | | | | | | |
| ²DT (25 h, | 1.44 | 1.38 | 43.43 | 64.72 | 1.37 | 1.00 |
| 10° C. | | | | | | |

¹Tensile strength and standard deviation (Nmm⁻²) dosed after 25 hours mixing and drying at 10° C.
²Disintegration time (seconds) dosed after 25 hours mixing and drying at 10° C.

It will be seen from Table 5 that the disintegration times are considerably reduced as compared with dosage forms which do not contain added acid without significant effect on tensile strength.

### Industrial Applicability

Solid, fast-dispersing pharmaceutical dosage forms have many desirable attributes. However, they also have various shortcomings. The present invention provides a method of production that solves the problems of increased disintegration times when high loadings of low water-solubility drugs are used. This discovery advances the state of the art in the preparation of fast-dispersing pharmaceutical dosage forms.

In the foregoing, there is provided a detailed description of the preferred embodiments of the present invention for the purpose of illustration and not limitation. It is to be understood that all other modifications, ramifications and equivalents obvious to those having skill in the art based on this disclosure are intended to be within the scope of the invention as claimed.

We claim

1. A method for shortening the disintegration time of a solid, fast-dispersing, lyophilized, pharmaceutical dosage form containing a substantially water-insoluble pharmaceutically active ingredient and a gelatin-based, water-dispersible carrier, said method comprising the step of including at least one edible acid in a composition containing said substantially water-insoluble pharmaceutically active ingredient and said gelatin-based, water-dispersible carrier, prior to formation of said solid dosage form from said composition.

2. The method as claimed in claim 1, wherein said at least one edible acid is selected from citric acid, maleic acid, tartaric acid and hydrochloric acid and mixtures of any one or more of such acids

3. The method as claimed in claim 1, further comprising the step of including at least one further carrier-forming component selected from sugars, cyclic sugars, inorganic salts, and amino acids.

4. The method as claimed in claim 3, wherein said at least one further carrier-forming component is mannitol.

5. The method as claimed in claim 1, wherein said composition is a suspension in which more than 50% by weight of the pharmaceutically active ingredient is in suspended form.

6. The method as claimed in claim 1, wherein said pharmaceutically active ingredient is present in an amount of at least 10% by weight of said solid dosage form.

7. The method as claimed in claim 1, wherein said at least one edible acid is present in an amount such as to produce a disintegration time of less than 10 seconds.

8. The method as claimed in claim 1, wherein the pharmaceutically active ingredient is selected from the group considting of paracetamol, piroxicam and rofecoxib.

9. The method as claimed in claim 1, wherein the pharmaceutically active ingredient is rofecoxib.

10. A method for reducing the disintegration time of a solid, fast-dispersing, lyophilized, pharmaceutical dosage form containing a substantially water-insoluble pharmaceutically active ingredient and a gelatin-based, water-dispersible carrier, said method comprising the step of including at least one edible acid selected from citric acid, maleic acid, tartaric acid and hydrochloric acid and mixtures of any one or more of such acids, in a composition containing water, said substantially water-insoluble pharmaceutically active ingredient and said gelatin-based, water-dispersible carrier, prior to formation of said solid dosage form from said composition.

11. The method as claimed in claim 10, further comprising the step of including at least one further carrier-forming component selected from sugars, cyclic sugars, inorganic salts and amino acids, in said carrier composition.

12. The method as claimed in claim 11, wherein said at least one further carrier-forming component is mannitol.

13. The method as claimed in claim 10, wherein said composition is a suspension in which more than 50% by weight of the pharmaceutically active ingredient is in suspended form.

14. The method as claimed in claim 10, wherein said pharmaceutically active ingredient is present in an amount of at least 10% by weight of said solid dosage form.

15. The method as claimed in claim 10, wherein said at least one acid is present in an amount such as to produce a disintegration time of less than 10 seconds.

16. The method as claimed in claim 10, wherein the pharmaceutically active ingredient is selected from the group considting of paracetamol, piroxicam and rofecoxib.

17. The method as claimed in claim 10, wherein the pharmaceutically active ingredient is rofecoxib.

18. A method for shortening the disintegration time of solid, fast-dispersing, lyophilized, pharmaceutical dosage forms, said method comprising the steps of:-
(i) forming a composition comprising water, a substantially water-insoluble pharmaceutically active ingredient, a gelatin-based, water-dispersible carrier and at least one edible acid selected from citric acid, maleic acid, tartaric acid and hydrochloric acid and mixtures of any one or more of such acids;
(ii) introducing portions of said composition into individual pockets; and
(iii) lyophilizing said portions in said pockets so as to dry and solidify said portions whereby to produce said solid dosage forms which contain said substantially water-insoluble pharmaceutically active ingredient, said gelatin-based, water-dispersible carrier and said at least one edible acid.

19. A method for the preparation of a solid, fast-dispersing, lyophilized, pharmaceutical dosage form, said method comprising the steps of;-
(i) forming a composition comprising water, a substantially water-insoluble pharmaceutically active ingredient, a gelatin-based, water-dispersible carrier and at least one edible acid selected from citric acid, maleic acid, tartaric acid and hydrochloric acid and mixtures of any one or more of such acids;
(ii) introducing portions of said composition into individual pockets; and
(iii) lyophilizing said portions in said pockets so as to dry and solidify said portions whereby to produce said solid dosage forms which contain said substantially water-insoluble pharmaceutically active ingredient, and wherein said dosage forms a disintegration times less than the same dosage forms without the edible acid.

20. The use of an edible acid in an oral, fast-dispersing, lyophilized, pharmaceutical solid dosage form containing a substantially water-insoluble pharmaceutically active ingredient and a gelatin-based, water-dispersible carrier, for shortening the disintegration time of the solid dosage form.

21. The use as claimed in claim 20, wherein said edible acid is a pharmaceutically acceptable acid selected from citric acid, maleic acid, tartaric acid, hydrochloric acid, and mixtures of any two or more thereof.

22. The use as claimed in claim 20, wherein said carrier also contains at least one further carrier-forming component selected from sugars, cyclic sugars, inorganic salts, and amino acids.

23. The use as claimed in claim 20, wherein said at least one further carrier-forming component is mannitol.

24. The use as claimed in claim 20, wherein said composition is a suspension in which more than 50% by weight of the pharmaceutically active ingredient is in suspended form.

25. The use as claimed in claim 20, wherein said pharmaceutically active ingredient is present in an amount of at least 10% by weight of said solid dosage form.

26. The use as claimed in claim 20, wherein said acid is present in an amount such as to produce a disintegration time of less than 10 seconds.

27. The use as claimed in claim 20, wherein the pharmaceutically active ingredient is selected from the group considting of paracetamol, piroxicam and rofecoxib.

28. The use as claimed in claim 20, wherein the pharmaceutically active ingredient is rofecoxib.
